(19) United States
(12) Patent Application Publication
(10) Pub. No.: US 2025/0173117 A1
(43) Pub. Date: May 29, 2025

(54) NEXT Microphone

(71) Applicant: Keone Trask, Greenville, SC (US)

(72) Inventor: Keone Trask, Greenville, SC (US)

(21) Appl. No.: 18/958,444

(22) Filed: Nov. 25, 2024

Related U.S. Application Data
(60) Provisional application No. 63/603,452, filed on Nov. 28, 2023.

(51) Int. Cl. G06F 3/16; H04R 1/02; H04R 3/00

(52) U.S. Cl. CPC G06F 3/162; G06F 3/165; H04R 1/028; H04R 3/00; H04R 2420/07; H04R 2420/09; H04R 2430/01

(57) ABSTRACT

Provided is a NEXT microphone system for use with a touchscreen display, audio hub, or audio/video hub. The NEXT Microphone comprises a microphone with a wireless transmitter capable of transmitting wireless audio to a touchscreen display or an audio visual hub. The touchscreen display or hub comprises an integral computer with an operating system and digital signal processing module. The digital signal processing module is capable of receiving the microphone audio signal and transmitting through at least one speaker simultaneously with the touchscreen display or hub audio output.

This application claims priority to pending U.S. Patent Application No. 63/603,452 filed Nov. 28, 2023, which is incorporated by reference.

### BACKGROUND OF THE INVENTION

The present invention generally relates to cordless microphones and, more particularly, to a novel wireless microphone system and method for voice augmentation and presentation which is particularly suitable for use with a dedicated touchscreen device utilizing a 6000A+ panel or an interface such as a NEXT Hub.

Educational institutions have transitioned over the past two generations away from chalk board use, first to marker boards and now to touchscreen displays. These touchscreens comprise a digital display with internal touch sensors and an on-board computer for operation.

While the touchscreens have continued to improve, little has improved with voice amplification and integration systems. Some classrooms and board rooms use a public address (PA) system that comprises a microphone, amplifier, and speakers. Few have any type of PA system integrated into the audio system used by projectors, touchscreens, and the like.

When audio enhancement is present in the classroom, it has been scientifically proven to improve student retention. In addition, having an audio enhancement solution removes teacher “voice fatigue” always present during the first few weeks of school. Unfortunately, adding an audio enhancement solution to every classroom is expensive. Although the benefits are widely recognized, it requires significant funding to place audio enhancement in every average size classroom.

What is needed is a complete upgrade solution that will integrate with interactive flat panels and/or interactive teaching devices. It also needs to be a low-cost device so that one can be purchased for every classroom.

### SUMMARY OF THE INVENTION

The present invention, referred to as the NEXT Microphone, provides a complete system and method for integrating a wireless microphone into an dedicated touchscreen device or modern touchscreen panel. The invention comprises at least one wireless microphone which may be clipped, in typical fashion, to the presenter's shirt, worn around the speaker's neck with a collar adapter, or held like a standard microphone.

In a particularly preferred embodiment the NEXT Microphone is interfaced with a Universal Serial Bus (USB) adapter that connects to the dedicated touchscreen device such as an 6000A+ Integrated Flat Panel (IFP) or a NEXT Hub.

A particular feature of the invention is the ability to utilize the internal speakers of a dedicated touchscreen device to become immediate output for the NEXT Microphone.

In a preferred embodiment the invention routes the microphone audio through a dedicated touchscreen device and out of the speaker amplification system. The invention can do the same with common audio hubs and works best with a NEXT Hub.

A preferred embodiment of the invention operates with the latest 6000A+ series IFPs or a NEXT Hub utilizing an onboard Rock Chip 3588.

The instant invention preferably utilizes an on-board chip and a USB receiver to allow for voice overlay and integration along with voice augmentation and enhancement.

The invention provides voice augmentation and enhancements to project the speaker's voice more intelligibly to the audience allowing the speaker to use a normal speaking voice and volume.

A particular advantage of the invention is realized in remote learning environments which is now essential in modern classrooms and boardrooms particularly post COVID pandemic wherein the appreciation of remote learning escalated.

The invention integrates the speaker's voice with the audio output of the IFP to provide a modern audio/visual learning experience, without the poor audio, video, computing, and connectivity issues experienced by many during the pandemic.

Yet another advantage of the invention is its small size allowing it to be placed in a suitcase for travel and then connected to any available 6000A+ IFP or NEXT Hub, thereby avoiding the need for shipment of a touchscreen device or PA system, while simultaneously providing users the latest audio/video capabilities while traveling.

Another advantage of the invention is that it allows for use of the NEXT Hub, which provides exceptional audio/video connectivity and clarity in boardrooms, classrooms, auditoriums and the like.

These and other advantages, as will be realized, are provided in a NEXT microphone system for use with a touchscreen display. The NEXT Microphone comprises a microphone comprising a wireless transmitter capable of transmitting a wireless link, wherein the wireless link comprises a microphone audio signal and a wireless receiver capable of receiving the wireless link and transmitting a line out signal to a touchscreen display, wherein the line out signal comprises the microphone audio signal. The touchscreen display comprises an integral computer comprising an operating system in functional control of the integral computer wherein the operating system comprises a digital signal processing module. The touchscreen display comprises at least one speaker for transmitting a touchscreen audio output. The digital signal processing module is capable of receiving the microphone audio signal from the line out signal and transmitting the microphone audio signal through at least one speaker simultaneously with the touchscreen audio output.

Yet another embodiment is provided in a NEXT microphone system for use with a touchscreen display comprising a microphone comprising a wireless transmitter capable of transmitting a wireless link wherein the wireless link comprises a microphone audio signal. A NEXT Hub is provided comprising an integral computer comprising an operating system in functional control of the integral computer wherein the operating system comprises a digital signal processing module. A wireless receiver is provided which is capable of receiving the wireless link and transmitting the microphone audio signal to the touchscreen display. The touchscreen display comprises at least one speaker for transmitting a touchscreen audio output and the digital signal processing module transmits the microphone audio signal through at least one speaker simultaneously with the touchscreen audio output.

Yet another embodiment is provided in a NEXT microphone system for use with a touchscreen display comprising a microphone comprising a wireless transmitter capable of transmitting a wireless link wherein the wireless link comprises a microphone audio signal. A NEXT Hub is provided comprising an integral computer comprising an operating system in functional control of the integral computer wherein the operating system comprises a digital signal processing module. A wireless receiver is provided which is capable of receiving the wireless link and transmitting the microphone audio signal to the touchscreen display. The touchscreen display comprises at least one speaker for transmitting a touchscreen audio output. The digital signal processing module transmits the microphone audio signal through at least one speaker simultaneously with the touchscreen audio output. The NEXT Hub provides for upgrade of legacy touchscreen digital displays by using existing touch process inputs and outputs of the legacy touchscreen digital displays.

### DESCRIPTION OF THE DRAWINGS

The invention will be more readily understood with reference to the following specification in conjunction with the drawings herein:

FIG. 1 is a schematic representation of an embodiment of the invention.

FIG. 2 is a flow-chart representation of a NEXT Hub which is an integral part of an embodiment of the invention.

FIG. 3 is a flow-chart representation of a NEXT Hub which is an integral part of an embodiment of the invention.

FIG. 4 is a flow-chart representation of a NEXT Hub which is an integral part of an embodiment of the invention.

FIG. 5 is a schematic front view representation of an embodiment of the invention shown as an USB hub.

FIG. 6 is a schematic rear view representation of an embodiment of the invention shown as an USB hub.

FIG. 7 is a schematic perspective view representation of a hand-held receiver for the USB hub embodiment of the invention.

FIG. 8 is a schematic perspective view representation of a hand-held receiver for the USB hub embodiment of the invention.

FIG. 9 is a schematic side view representation of an over-the-neck holder for use with the USB embodiment of the invention.

FIG. 10 is a schematic representation of an embodiment of the invention indicating a magnetic loop to an adapter cable between the USB receiver and an audio visual input.

### DESCRIPTION OF THE PREFERRED EMBODIMENTS

The present invention relates to a NEXT Microphone, which is particularly suitable for use with a dedicated touchscreen device such as an interactive flat panel (IFP) or when interfaced through a NEXT Hub. More specifically, the present invention is related to a NEXT Microphone which allows for audio transmission through a dedicated touchscreen device wherein the audio from the NEXT Microphone can be augmented for volume or special effects such as overlay over audio emanating from a connected IFP.

A preferred embodiment of the invention operates with the latest 6000A+ series IFPs and particularly the NEXT Hub, both of which utilize a Rock Chip 3588 as an on-board chip. The on-board chip in concert with a USB receiver allows for voice overlay and integration along with voice augmentation and enhancement.

The voice augmentation and enhancements project the speaker's voice more intelligibly to the audience. Studies have shown that such voice augmentation and enhancement improves student comprehension because of the improved audio. The teacher's voice is more intelligible so students have a greater understanding of the subject matter. Studies have also shown that this greatly improves student retention. The invention also reduces vocal fatigue by allowing the speaker to use a normal voice and volume, rather than yelling.

In the prior art, if audio were to be routed through mounted or recessed speakers, the microphone/PA system would need a direct connection which is separate from the audio of the display. Without separation, audio/video output and microphone/voice output would be in conflict for the same amplification system. With the present invention, all the audio, video, touch sounds, and microphone input is routed through the USB receiver. The USB receiver allows for resolution of conflicts, thereby allowing audio output to occur simultaneously. A video can play and the presenter can talk, even allowing the audio of the video to be subservient in volume relative to the audio through the NEXT Microphone, such as of a speaker or lecturer, so the audio through the NEXT Microphone can take preeminence. This is also called audio ducking.

Audio ducking is preferably a function handled within a NEXT Hub. A digital signal processing (DSP) module integral to the NEXT Hub detects audio input from the line-out signal on the USB receiver. When this input is detected, the DSP module reduces the audio from the operating system of the NEXT Hub, allowing the NEXT Microphone to have priority with regards to volume and augmentation relative thereby allowing for audio ducking.

A particular problem associated with the prior art, particularly in audio ducking applications, is audio feedback. In an embodiment of the invention this is resolved by adding a magnetic loop to an adapter cable between the USB receiver and an audio visual input.

The invention will be described with reference to the figures which are an integral part of the instant disclosure provided for clarification of the description of the invention without limit thereto. As appropriate, similar elements will be numbered accordingly.

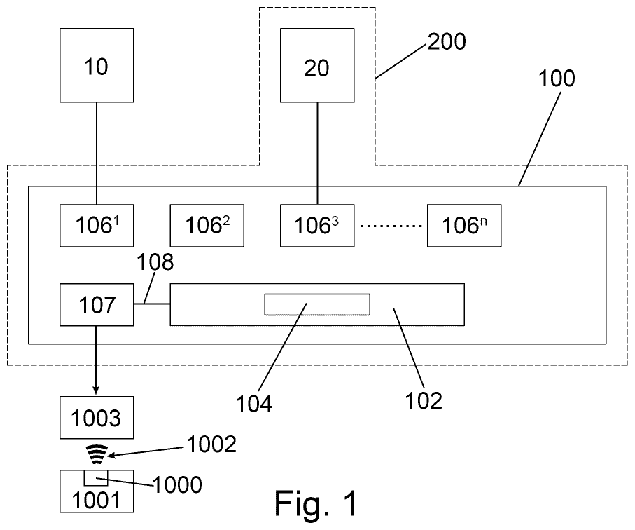

An embodiment of the invention is illustrated in schematic view in FIG. 1. In FIG. 1, a NEXT Hub, 100, comprises an integral computer, 102, with an operating system, 104, in functional control of the integral computer. Input/Output (I/O) jacks, 106, are in signal communication with the integral computer wherein each I/O jack is capable of signal communication with at least one dedicated touchscreen device, 10, and an optional touchstone digital display, 20. At least one I/O jack is a USB Receiver, 107. A device for receiving an audio signal NEXT Microphone, 1001, comprises a wireless transmitter, 1000, which transmits a wireless link, 1002, which is received by a wireless receiver, 1003, which is integral to or input to the USB Receiver. When a signal is received by the USB receiver from the NEXT Microphone the NEXT Hub or IFP detects audio input in the form of a line out signal, 108. A digital signal processing (DSP) module integral to the NEXT Hub or IFP detects the audio input from the line out audio signal from the USB receiver and reduces the audio from the operating system allowing the NEXT Microphone to have priority, thereby causing the audio signal from the integral computer to be lower, thereby audio ducking the audio signal from the NEXT Microphone. In one embodiment, the NEXT Hub may be integral to touchstone digital display, 20, as indicated by 200.

Wireless links are common in the art and not particularly limited herein. Wireless links utilizing a signal over a carrier wave are well known and include Bluetooth and other wireless links carried over an ultrahigh frequency carrier wave as defined, for example, in legacy standard IEEE 802.15.1 and modernized equivalents thereof.

Though not limited thereto, I/O jacks in signal communication with the dedicated touchscreen device are referred to as device I/O jacks, and I/O jacks in signal communication with the touchscreen digital display are referred to as display I/O jacks for the purposes of clarity of discussion.

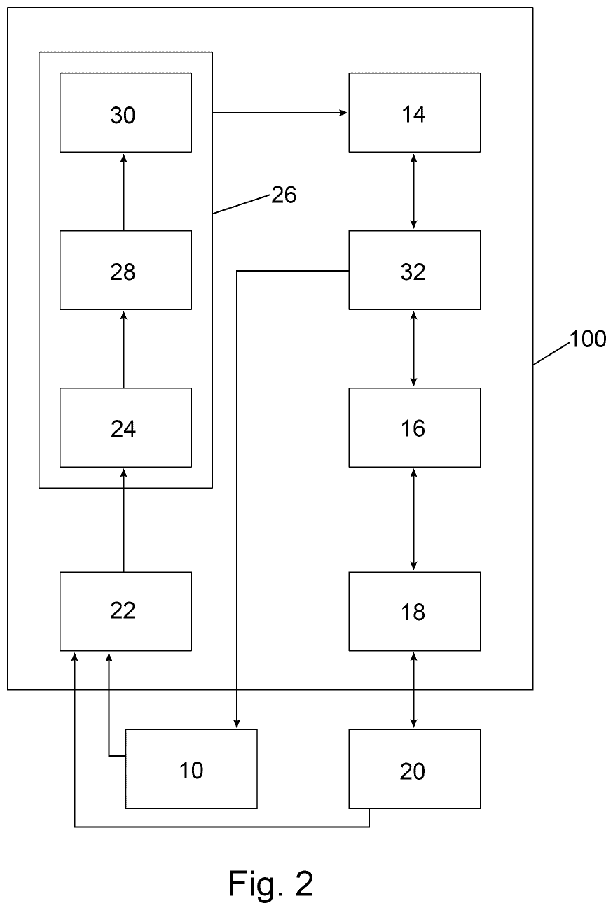

A NEXT Hub will be described with reference to FIG. 2, wherein the architecture of the NEXT Hub, 100, is illustrated in a flow chart representation. In FIG. 2, a dedicated touchscreen device, 10, is in signal communication, preferably touch communication, with a multitouch module switch, 22. As will be realized, the multitouch module allows for multiple inputs from different sources. The multitouch module switch is in signal communication, preferably touch communication, with the input system, 24, of the an integral computer comprising an operating system, 26. As would be realized, the input system of the operating system has integral therewith an input reader, 28, and dispatch module, 30, which provides signal output to the touch process module, 14. The touch process module creates a touch process command which is transmitted to a touch process switch, 32. The touch process command is transmitted to the dedicated touchscreen device, 10, and to the output driver, 16, for simultaneous display of the same image on a dedicated touchscreen device and a touchscreen digital display through the touch-out port, 18. Signal communication can include audio, video or image control commands. Touch communication is signal communication comprising touch commands from a digital touch device such as a dedicated touchscreen device or a touchscreen digital display such as an IFP.

With further reference to FIG. 2, in a parallel process the touchscreen digital display is also in signal communication with the multitouch module switch, 22, which is in signal communication with the operating system, 26, of the display computer. As would be realized from the discussion herein, the operating system, 26, allows any device which is compatible with the operating system to interface with any other compatible device. By switching the signal input at the multitouch module switch, 22, and the touch process switch, 32, the results of touch commands can be simultaneously manipulated by touch command on either the dedicated touchscreen device or the touchscreen digital device and displayed on both simultaneously.

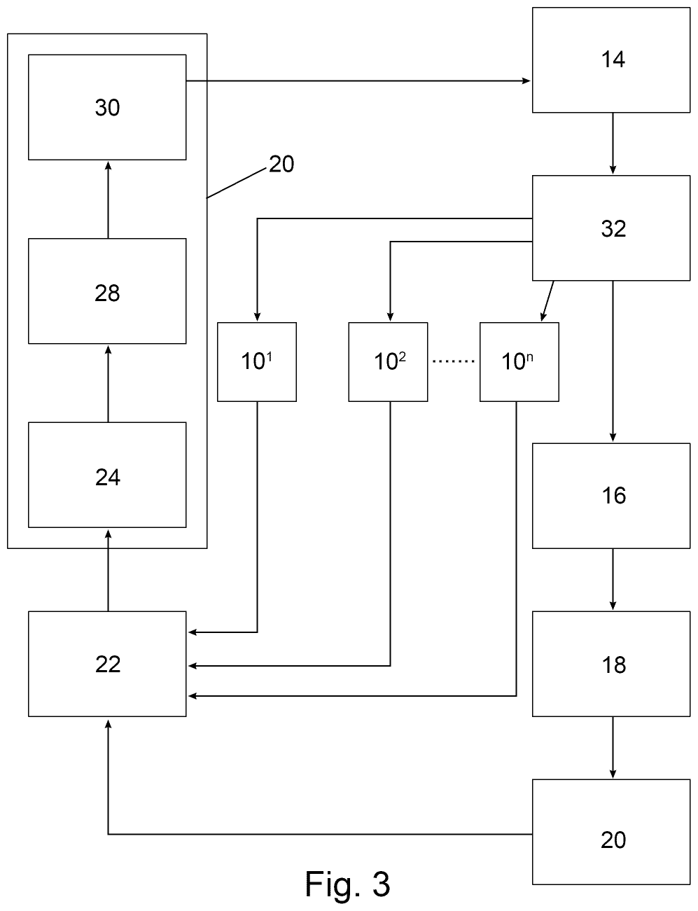

An embodiment of the NEXT Hub is illustrated schematically in FIG. 3 wherein multiple dedicated touchscreen devices, 10¹-10ⁿ, are illustrated. The number of dedicated touchscreen devices, represented by superscript n, is not particularly limited by the invention but only by the availability of dedicated touchscreen devices and switch capacity.

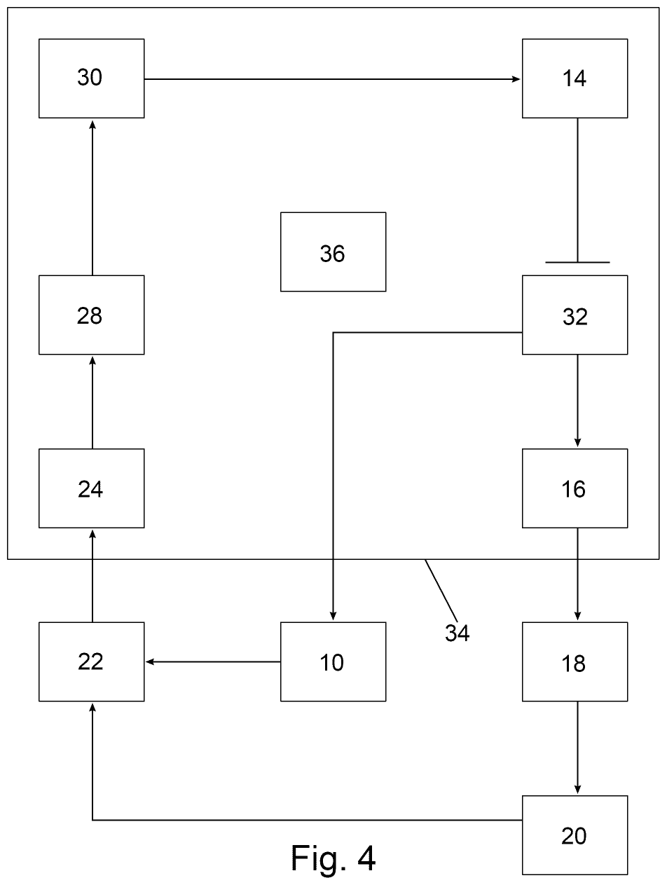

FIG. 4 also illustrates an embodiment of the NEXT Hub. In FIG. 4, an additional layer of the operating system, 34, comprises an application, 36, wherein the application, referred to in the art as an app, allows for additional functionality on the touchscreen digital display. Layered architecture, the implementation of which is well known in the art, allows for the use of tools include screensharing, annotation, screenshot capabilities, interactive lessons, device management, and collaborating work.

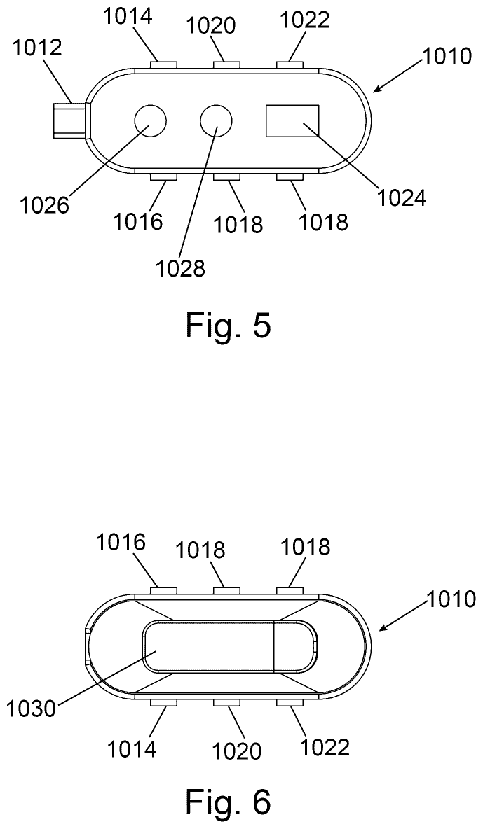

An embodiment of the NEXT Microphone is illustrated schematically in FIG. 5 wherein the NEXT Microphone, 1010, is illustrated in side view. The NEXT Microphone comprises a, preferably male, USB connector, 1012, which is preferably retractable for storage and transport convenience. A retractor, 1014, extends and retracts the USB connector as would be well understood in the art and not necessarily limited herein. A power switch, 1016, allows the NEXT Microphone to be powered on and off as would be well understood to those of skill in the art. Volume buttons, 1018, for increasing and decreasing volume would be provided as well known in the art and not necessarily limited herein. An input port, 1020, allows for connectivity to an auxiliary charge cord or for input of auxiliary audio signal, such as music, wherein the auxiliary audio signal is transmitted in addition to, or in lieu of, voice related audio signals. A control switch, 1022, is provided for connectivity to a wireless receiver such as by bluetooth and the like. The NEXT Microphone is normally powered by the USB connection from the attached device. It may also include onboard power storage, 1024, such as a battery, to provide power to the circuitry, not shown, and microphone, 1026, and transmitter, 1028, wherein the transmitter transmits the wireless signal to the wireless receiver.

An embodiment of the NEXT Microphone, 1010, is illustrated in schematic back view in FIG. 6 wherein the USB connector is retracted and not visible. A clip, 1030, allows the NEXT Microphone to be placed on a collar, lapel, hat, or other article of clothing, or as a clothing accessory such as a pendant.

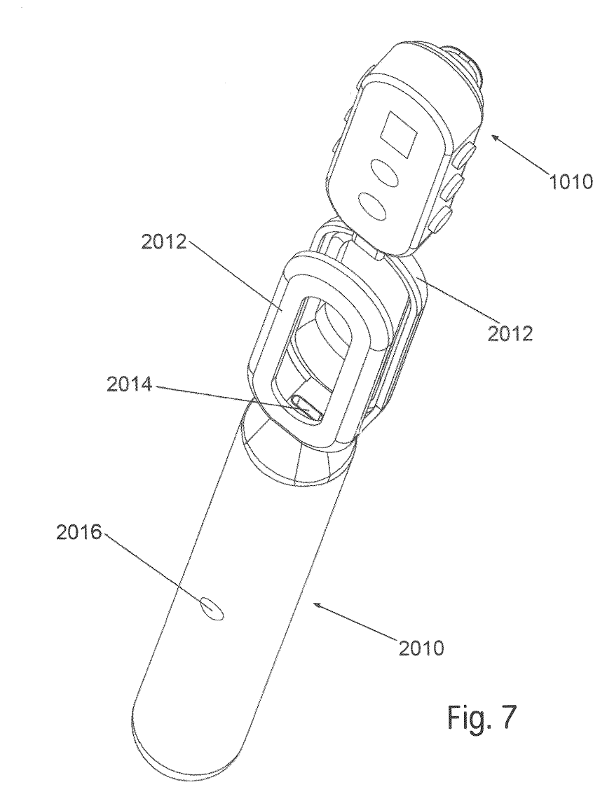

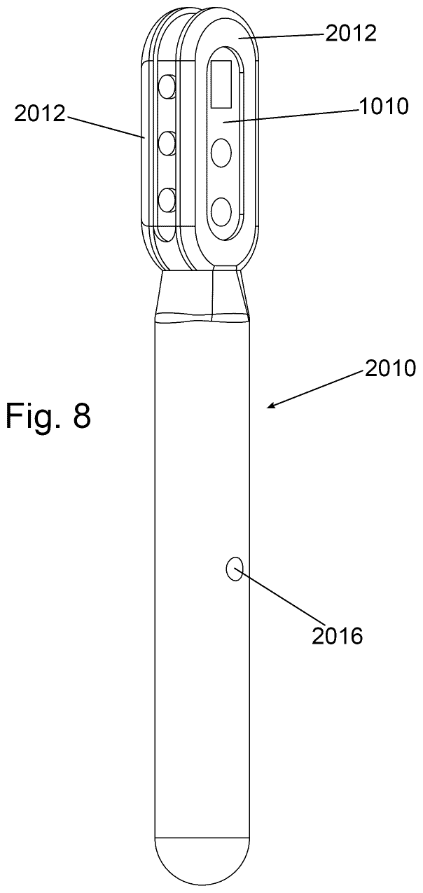

FIG. 7 illustrates an optional hand-held device for receiving an embodiment of the invention. In FIG. 7 a handle, 2010, is illustrated in schematic representation wherein the handle is capable of holding the NEXT Microphone, 1010. The handle comprises adjacent leafs, 2012, which are preferably separable in opposing directions against a bias towards closure around a NEXT Microphone constrained therein as illustrated schematically in FIG. 8. The handle preferably has a USB port, 2014, capable of mating with the USB connector of the NEXT Microphone. A particular feature of the handle is an energy source, such as a handle battery, which is preferably rechargeable through a charging port, 2016, wherein the NEXT Microphone is recharged by the handle battery through an integrated connector, such as a mated USB connector. The handle allows the NEXT Microphone to be optionally used as a handheld device similar to a conventional microphone.

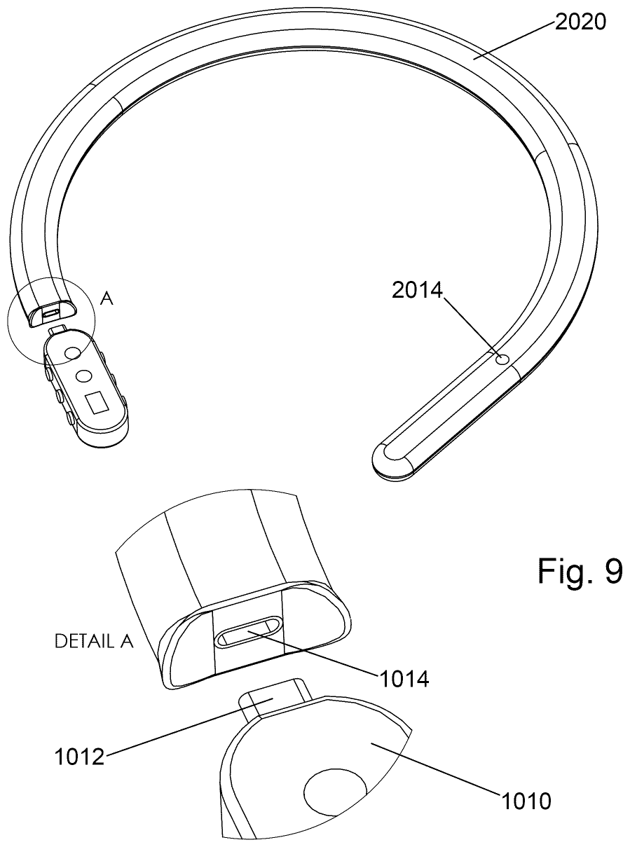

FIG. 9 illustrates another embodiment of the invention in schematic view. In FIG. 9, a handle is provided in an arcuate shape to form a neck collar for the NEXT Microphone. The neck collar, 2020, comprises a USB port, 2014, capable of mating with the USB connector of the NEXT Microphone. A particular feature of the neck collar is an energy source, such as a collar battery, which is preferably rechargeable through a charging port, 2014, wherein the NEXT Microphone is powered and recharged through the mated USB connectors by the collar battery. The neck collar can be worn around the neck of the user thereby allowing the microphone to be maintained in a comfortable position without the use of the hands and in an optimal position for picking up the voice of the user.

The NEXT microphone may be used with most modern IFPs and other audio visual hubs but is particularly well suited for use with a NEXT Hub, which comprises another aspect of the invention. The integral computer of the NEXT Hub preferably includes a 6000A+ series on-board chip that is capable of operation with all modern audio and video devices. A particularly preferred embodiment of the NEXT Hub comprises a 4k camera and a 6-array microphone to provide state of the art video capabilities for any touchscreen.

A particular advantage of the NEXT Hub is the ability to utilize different operating systems. Preferred operating systems include Windows loaded on the Intel Pluggable Specification (OPS), Smart Display Module (SDM), macOS or ChromeOS. Most preferably the operating system is Windows loaded on the Intel Pluggable Specification (OPS).

The NEXT Hub preferably comprises speakers thereby allowing full functionality as a sound bar. Most preferred are high-fidelity, high-volume amplifiers and speakers which provide clarity in larger environments such as those common in classrooms and boardrooms. Speakers which are 6-array speakers are preferable. Speakers coupled with an audio amplifier of at least 15 watts are preferable.

Connectivity is not particularly limited by the NEXT Hub. Particularly preferred are USB-A, USB-C, HDMI, VGA and other conventional cables due to widespread availability.

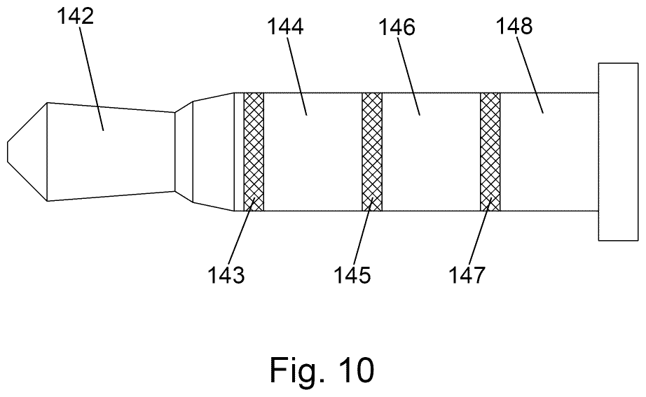

Typical IFPs and soundbars cannot receive a microphone input from an 4-pole TRRS jack and output the audio from the speaker. Conventional 4-pole TRRS jacks, illustrated schematically in FIG. 10 as 140, comprise a tip, 142, a first ring, 144, separated from the tip by an insulator ring, 143, a second ring, 146, separated from the first ring by a second insulator ring, 145, and a sleeve, 148, separated by the second ring by a third insulator ring, 147. In conventional use, the 4-pole TRRS jack tip is speaker left output, the first ring is speaker right output, the third ring is electrical ground and the sleeve is microphone input. To achieve one embodiment of the instant invention, the conventional connectivity of the 4-pole TRRS jack tip is speaker left output, the first ring is speaker right output, the third ring is microphone and the sleeve is electrical ground. This allows speaker input to be utilized while bypassing conventional circuitry. As would be realized, resistance is provided between left and right speaker input and electrical ground and between microphone input and electrical ground.

To provide further functionality, this embodiment of the invention even allows connection of the OPS computer from an IFP or a separate laptop. In this way, all former and current computing functionality is provided through the invention. For example, when connected in this manner, this embodiment of the invention allows for screen capturing, annotation, and touchscreen capability, using the existing glass screen, but all managed by the upgraded computer in the invention.

In a preferred embodiment, the NEXT Hub utilizes the chipset, software, and EDLA Android 13 ecosystem of 6000A+ panels. This means older touchscreens panels can get a software update, OS update, and feature set upgrade all within a device that is far more economical than full replacement of the touchscreen. It will also save on E-waste/disposal costs of old technology. Particularly preferred chips for interface with touchscreen devices are RK3588 available commercially from Rockchip Electronics Co. LTD.

This embodiment of the invention provides the latest software suite to all users. Prior art touchscreens typically did not provide for software upgrades and, when they did, the upgrades were time-consuming and cumbersome. The NEXT Hub is Internet connected and allows for seamless upgrades.

The shape and size of the inventive NEXT Hub is not particularly limited with the exception of having sufficient size to allow for the desired number of I/O jacks and, if employed, sufficient facial area for speaker performance. A particular embodiment is sufficiently small to fit into a conventional suitcase for travel. This allows the NEXT Hub to be mobile and connectible to any available video screen upon arrival, avoiding the need for shipment of a modern touchscreen, while simultaneously providing users the latest audio/video capabilities while traveling.

This aspect of the present invention is compatible with Enterprise Device Licensing Agreement (EDLA) certification requirement which allows for 16 points of touch. EDLA is a collaboration between Google and Android developers to certify a device according to a set of standards. Achieving EDLA has become a standard in and of itself for the IFP industry allowing for the panels to have a complete review by a third party. EDLA certification of the NEXT Hub allows older touchsource digital displays to be updated to a newer operating system, such as Android OS without prior vulnerabilities.

A touchscreen display manufacturer may provide many proprietary applications and functions that require a specific match between hardware and software, so they design and manufacture their own OPS computers to maximize the functionality of their touchscreens. If a user were to upgrade with a generic OPS computer, the result may be reduced functionality or even complete system failure due to mismatched hardware and software. Unscrupulous users may even upgrade a touchscreen display with a faster but less compatible computer and then attempt to pass it off as a newer model for sale.

In some cases, therefore, the solution is to upgrade the computer using a proprietary adapter to install a new OPS computer. But, where the touchscreen is very old or simply has little to no functionality beyond the glass itself, simply upgrading the computer only provides part of the solution.

Touch points are the number of touch points a screen can recognize simultaneously. A single touch point, for example, would only allow for simple control such as tap, scroll or swipe using a single stylus or finger, for example. With more simultaneous touch points more complex actions can be used such as multiple-finger scrolling, multiple finger expand or zoom, and more complex actions involving multiple fingers, or stylus points, on the screen simultaneously. The number of touch points is not particularly limited herein with the understanding that advances in the number of touch points on a screen will likely continue to grow and therefore the NEXT Hub allows compatibility with increased touch points which was previous difficult to provide.

Elements of the NEXT Hub not otherwise specified are well known in the art and not necessarily limited herein including memory storage, touch screen technology, power systems and the like. A particular feature of this aspect of the instant invention is the ability to interface and function with any system compatible with the generic operating system used such as Intel OPS.

Once the USB Receiver is connected to the device, all audio from the display is then routed through the Receiver including: the microphone, android OS audio, PC module audio, and external devices. The invention routes audio from the microphone through the audio system of the display, which allows the speaker's voice to “duck” audio already playing on the display. This means that a teacher, for example, may play an educational video on the display and then speak over the audio from that video in key portions to point out significant facts.

The wireless microphone already has a “clip” that allows it to be clipped to the presenters clothes. The microphone can also attached to a Collar adapter to be worn around the neck or connected to a handheld adapter. Again, the USB Receiver must be connected to a modern IFP, preferably one using a 6000A+ panel or the NEXT Hub.

The invention has been described with reference to the preferred embodiments without limit thereto. Those of skill in the art may realize additional improvements and embodiments which are not specifically stated but which are within the scope of the invention as set forth in the claims appended hereto.

1. A NEXT microphone system for use with a touchscreen display comprising:
a microphone comprising a wireless transmitter capable of transmitting a wireless link, wherein said wireless link comprises a microphone audio signal;
a wireless receiver capable of receiving said wireless link and transmitting a line out signal to a touchscreen display, wherein said line out signal comprises said microphone audio signal;
wherein said touchscreen display comprises an integral computer, comprising an operating system in functional control of said integral computer, wherein said operating system comprises a digital signal processing module, and said touchscreen display comprises at least one speaker for transmitting a touchscreen audio output; and
wherein said digital signal processing module is capable of receiving said microphone audio signal from said line out signal and transmitting said microphone audio signal through said at least one speaker simultaneously with said touchscreen audio output.

2. The NEXT microphone system of claim 1 wherein said digital signal processing module lowers a screen volume of said touchscreen audio output relative to a microphone volume of said microphone audio signal.

3. The NEXT microphone system of claim 1 wherein said wireless receiver operatively connects to said touchscreen display using a 6000A+ computer architecture.

4. The NEXT microphone system of claim 1 further comprising a connector.

5. The NEXT microphone system of claim 4 wherein said connector is a USB.

6. The NEXT microphone system of claim 4 wherein said connector mates with a connector of a handle.

7. The NEXT microphone system of claim 6 wherein handle has an arcuate shape.

8. The NEXT microphone system of claim 1 further comprising an adapter capable comprising a magnetic loop.

9. The NEXT microphone system of claim 1 wherein said wireless receiver connects to said touchscreen display by a plug.

10. The NEXT microphone system of claim 9 wherein said plug comprises a jack tip electrically connected to speaker left output, a first ring electrically connected to speaker right output, a third ring electrically connected to said microphone and a sleeve electrically connected to electrical ground.

11. A NEXT microphone system for use with a touchscreen display comprising:
a microphone comprising a wireless transmitter capable of transmitting a wireless link, wherein said wireless link comprises a microphone audio signal;
a NEXT Hub comprising:
an integral computer comprising an operating system in functional control of said integral computer wherein said operating system comprises a digital signal processing module; and
a wireless receiver capable of receiving said wireless link and transmitting said microphone audio signal to said touchscreen display;
wherein said touchscreen display comprises at least one speaker for transmitting a touchscreen audio output and wherein said digital signal processing module transmits said microphone audio signal through said at least one speaker simultaneously with said touchscreen audio output.

12. The NEXT microphone system of claim 11 further comprising a connector.

13. The NEXT microphone system of claim 12 wherein said connector is a USB.

14. The NEXT microphone system of claim 12 wherein said connector mates with a connector of a handle.

15. The NEXT microphone system of claim 14 wherein handle has an arcuate shape.

16. The NEXT microphone system of claim 11 further comprising an adapter capable comprising a magnetic loop.

17. The NEXT microphone system of claim 11 wherein said wireless receiver connects to said touchscreen display by a plug.

18. The NEXT microphone system of claim 17 wherein said plug comprises a jack tip electrically connected to speaker left output, a first ring electrically connected to speaker right output, a third ring electrically connected to said microphone and a sleeve electrically connected to electrical ground.

19. The NEXT microphone system of claim 11 wherein said NEXT Hub further comprises:
at least one device I/O jack capable of receiving incoming signal communication from said integral computer;
at least one display I/O jack capable of sending outgoing signal communication to said integral computer;
a multitouch module switch capable of being in signal communication with said device I/O and said display I/O;
wherein said operating system is capable of receiving a touch communication from said multitouch module switch and wherein said operating system converts said touch communication to a touch process command; and
a touch process switch capable of receiving said touch process command and transmitting said touch process command to said display I/O or said device I/O.

20. The NEXT microphone system of claim 19 further comprising a digital touch device in signal communication with at least one of said display I/O or said device I/O.

21. The NEXT microphone system of claim 20 wherein said digital touch device is a touchscreen digital display.

22. The NEXT microphone system of claim 20 wherein said digital touch device is a dedicated touchscreen device.

23. The NEXT microphone system of claim 19 wherein said operating system is selected from the group consisting of Intel Pluggable Specification and Smart Display Module.

24. The NEXT microphone system of claim 23 wherein said operating system is Intel Pluggable Specification.

25. The NEXT microphone system of claim 19 wherein said operating system further comprising an application layer.

26. The NEXT microphone system of claim 25 wherein said application layer is selected from the group consisting of screensharing, annotation, screenshot, interactive lessons, device management, and collaborating work.

27. The NEXT microphone system of claim 19 wherein at least one of said device I/O or said display I/O is a connected selected from the group consisting of USB-A, USB-C, HDMI and VGA.

28. The NEXT microphone system of claim 19 further comprising a video camera.

29. A NEXT microphone system for use with a touchscreen display comprising:
a microphone comprising a wireless transmitter capable of transmitting a wireless link wherein said wireless link comprises a microphone audio signal;
a NEXT Hub comprising:
an integral computer comprising an operating system in functional control of said integral computer wherein said operating system comprises a digital signal processing module; and
a wireless receiver capable of receiving said wireless link and transmitting said microphone audio signal to said touchscreen display;
wherein said touchscreen display comprises at least one speaker for transmitting a touchscreen audio output and wherein said digital signal processing module transmits said microphone audio signal through said at least one speaker simultaneously with said touchscreen audio output; and
wherein said NEXT Hub provides for upgrade of legacy touchscreen digital displays by using existing touch process inputs and outputs of said legacy touchscreen digital displays.

30. The NEXT microphone system of claim 29 further comprising a connector.

31. The NEXT microphone system of claim 30 wherein said connector is a USB.

32. The NEXT microphone system of claim 30 wherein said connector mates with a connector of a handle.

33. The NEXT microphone system of claim 32 wherein handle has an arcuate shape.

34. The NEXT microphone system of claim 29 further comprising an adapter capable comprising a magnetic loop.

35. The NEXT microphone system of claim 29 wherein said wireless receiver connects to said touchscreen display by a plug.

36. The NEXT microphone system of claim 35 wherein said plug comprises a jack tip electrically connected to speaker left output, a first ring electrically connected to speaker right output, a third ring electrically connected to said microphone and a sleeve electrically connected to electrical ground.

37. The NEXT microphone system of claim 29 wherein said NEXT Hub further comprises:
at least one device I/O jack capable of receiving incoming signal communication from said integral computer;
at least one display I/O jack capable of sending outgoing signal communication to said integral computer;
a multitouch module switch capable of being in signal communication with said device I/O and said display I/O;
wherein said operating system is capable of receiving a touch communication from said multitouch module switch and wherein said operating system converts said touch communication to a touch process command; and
a touch process switch capable of receiving said touch process command and transmitting said touch process command to said display I/O or said device I/O.

38. The NEXT microphone system of claim 37 further comprising a digital touch device in signal communication with at least one of said display I/O or said device I/O.

39. The NEXT microphone system of claim 37 wherein said digital touch device is a touchscreen digital display.

40. The NEXT microphone system of claim 37 wherein said digital touch device is a dedicated touchscreen device.

41. The NEXT microphone system of claim 37 wherein said operating system is selected from the group consisting of Intel Pluggable Specification and Smart Display Module.

42. The NEXT microphone system of claim 41 wherein said operating system is Intel Pluggable Specification.

43. The NEXT microphone system of claim 37 wherein said operating system further comprising an application layer.

44. The NEXT microphone system of claim 43 wherein said application layer is selected from the group consisting of screensharing, annotation, screenshot, interactive lessons, device management, and collaborating work.

45. The NEXT microphone system of claim 37 wherein at least one of said device I/O or said display I/O is a connected selected from the group consisting of USB-A, USB-C, HDMI and VGA.

46. The NEXT microphone system of claim 37 further comprising a video camera.
